(19) United States
(12) Patent Application Publication
(10) Pub. No.: US 2006/0149127 A1
(43) Pub. Date: Jul. 6, 2006

(54) Disposable multi-lumen catheter with reusable stylet

(76) Inventors: Fred R. Seddiqui, Los Altos, CA (US); Alex C. Niel, Daly City, CA (US); John Higgins, Los Altos, CA (US); Rupesh K. Desai, San Jose, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/153,007

(22) Filed: Jun. 14, 2005

Related U.S. Application Data
(63) Continuation-in-part of application No. 11/026,432, filed on Dec. 30, 2004, now abandoned.

(51) Int. Cl. A61B 1/00

(52) U.S. Cl. 600/104

(57) ABSTRACT

A disposable catheter and reusable stylet assembly is disclosed. The disposable catheter prevents infectious bodily fluids and tissues from making contact with the stylet. The catheter includes an implantable elongated tube connected to a stylet receiving body. The stylet includes a control handle that can be coupled or engaged to the receiving body. The stylet also includes a stylet member that can extend within the catheter tube. An image capturing component can be positioned at the distal end of the stylet member. A control mechanism can control the movement of the stylet member.

### CROSS-REFERENCE

This application is a continuation-in-part of application Ser. No. 11/026,432 filed on Dec. 30, 2004.

### TECHNICAL FIELD

The present invention relates to a disposable multi-lumen catheter with a reusable stylet. More specifically, the present invention relates to a disposable catheter for isolating a stylet from virus, bacteria and bodily fluids and for allowing multiple diagnostic and/or therapeutic instrumentations to be used during a procedure.

### BACKGROUND

Implantable or insertable medical devices such as endoscopes are used for performing diagnostic and/or therapeutic procedures in a variety of bodily cavities. Upper GI endoscopes are used for esophagus, stomach, duodenum; colonoscopes for examination of colon; bronchoscopes for the bronchi; laparoscopes for peritoneal cavity; sigmoidoscopes for the rectum and the sigmoid colon; and angioscopes for the examination of blood vessels. In a procedure, an insertion length of the endoscope becomes contaminated with bodily tissues and fluid. Accordingly, once contaminated, the same scope cannot be successively used for another patient.

Once used, an endoscope must be subjected to a vigorous cleaning process which can be expensive and time consuming. In light of the fact the doctors have to perform multiple procedures, the idle time between the procedures for cleaning tends to be longer than the procedure itself, adding to the cost of the procedure. Additionally, more cost can be attributed to cleaning equipment and personnel. With the use of ethylene oxide as a cleaning agent, exposure of 24 hours is required. The gas is toxic and needs to be adequately removed from the device. Exposure of such plastic implantable medical devices to cleaning agents can also cause damage to the device. Gluteraldehyde solutions may be used for disinfection. However, the length of exposure should exceed one hour and still this may not lead to a thorough sterilization. Moreover, the chemical is toxic and must be rinsed carefully before the device is implanted or inserted into another patient. Residual cleaning agents on the device can cause adverse effects on a patient including irritation and inflammation.

The problem of adequate sterilization of an endoscope is further exacerbated by the fact that endoscopes include channels and lumens extending through the core of the device. These lumens are not readily accessible to cleaning gases and fluids and as a result, isolation of the endoscope from the patient is needed. In order to address this problem, endoscope systems which are protected by a disposable sheath having channels formed therein have been described in various patent literatures: U.S. Pat. Nos. 5,924,977, 4,721,097, 4,741,326, 4,825,850, 4,869,238, 4,911,564, 4,991,565, 5,050,585, and 4,646,722.

In addition to isolation of the endoscope from exposure to contaminants, an endoscope system should be able to perform multiple therapeutic and/or diagnostic functions, preferably at one time. For example, in some procedures, it may be advantageous to perform multiple functions of, for example, biopsy and drug delivery, balloon expansion and biopsy, or balloon expansion and drug delivery, all simultaneously. As a result, the protective cover should be constructed to allow for a physician to have the option of performing multiple procedures without the need to extract one therapeutic or diagnostic device in favor of insertion of another.

### SUMMARY

A disposable catheter and reusable stylet assembly is disclosed, comprising a catheter assembly and a stylet. The catheter assembly comprises a catheter tube having at least two lumens extending at least partially through the catheter tube. The catheter assembly further includes a stylet receiving body connected to the catheter tube. The receiving body includes an opening along a longitudinal side of the body and a port disposed within the receiving body and in fluid communication with at least one of the lumens. The stylet comprises a control handle capable of being securely inserted in the longitudinal side opening of the receiving body and a stylet member or tube for being extended through a lumen of the catheter assembly. The stylet can include a camera for capturing pictures or video images.

The catheter assembly prevents the stylet tube or the control handle from making contact with bodily fluids or tissues. Accordingly the reusable stylet is protected by a disposable catheter even if not properly sterilized.

### DESCRIPTION OF THE FIGURES

FIG. 1 is a perspective view of a stylet-catheter assembly in an assembled configuration;

FIG. 2 is a perspective view of the stylet-catheter assembly in a disassembled configuration;

FIG. 3 is a side view of an embodiment of a receiving body of the catheter assembly for receiving a control handle of the stylet;

FIG. 4 is a schematic of one embodiment of the receiving body of the catheter assembly, illustrating the lumens disposed within the body thereof;

FIG. 5 illustrates an embodiment of the distal tip of the catheter assembly with the stylet positioned therein;

FIG. 6 illustrates an embodiment of the distal tips of the catheter assembly and the stylet as disassembled;

FIG. 7 is a cross sectional view of an embodiment of the stylet disposed within the catheter assembly; and

FIGS. 8A, 8B and 8C are cross sectional views of embodiments of the stylet disposed within the catheter assembly.

### DETAIL DESCRIPTION OF THE EMBODIMENTS OF THE INVENTION

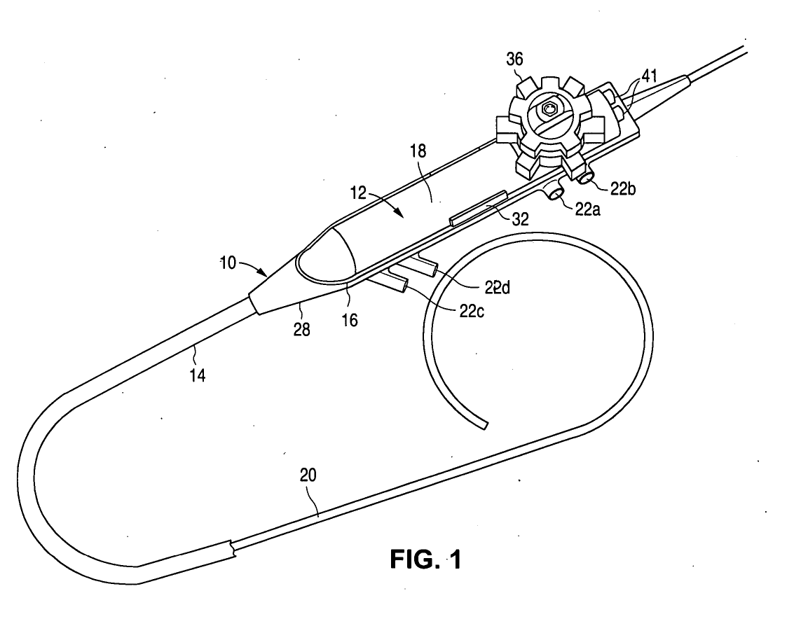

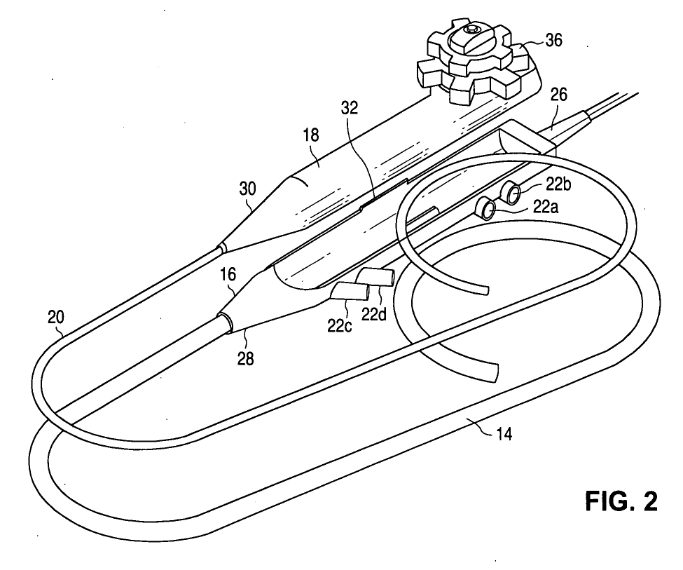

The embodiments of the present invention are directed to implantable or insertable medical devices for performing medical procedures such as diagnostic and/or therapeutic procedures. Implantable means that the device could remain in the body for any duration of time, preferably only a temporary duration of time such that the device is removed after its intended use. FIGS. 1 and 2 illustrate a disposable multi-lumen catheter assembly 10 with a reusable stylet 12. The stylet 12 can be releasably coupled to the catheter assembly 10. The catheter assembly 10 includes an elongated catheter tube or body 14 in connection with a stylet control handle receiving body 16. The elongated catheter tube or body 14 can be of any length, preferably as long as a steerable stylet member or tube 20. In FIG. 1, only a section of body 14 is illustrated so that tube 20 can be seen. The receiving body 16 is adapted to receive a reusable control handle 18 of the reusable stylet 12. In this configuration, the control handle 18 can be fittingly mated within the receiving body 16 while the steerable stylet member or tube 20 of the stylet 12 is disposed within a lumen of the elongated catheter body 14. It will be apparent that the stylet 12 will be fully protected by the disposable catheter body 14 such that if the stylet 12 is not adequately sterilized, a patient will be fully protected from exposure and infection, such as from gastrointestinal diseases, with the use of a new protective catheter assembly 10. In one embodiment, the stylet member 20 is disconnectable from the control handle 18. In this embodiment, both pieces or just the stylet member 20 can be disposable. In another embodiment, stylet member 20 is in fixed or permanent connection with the control handle 18.

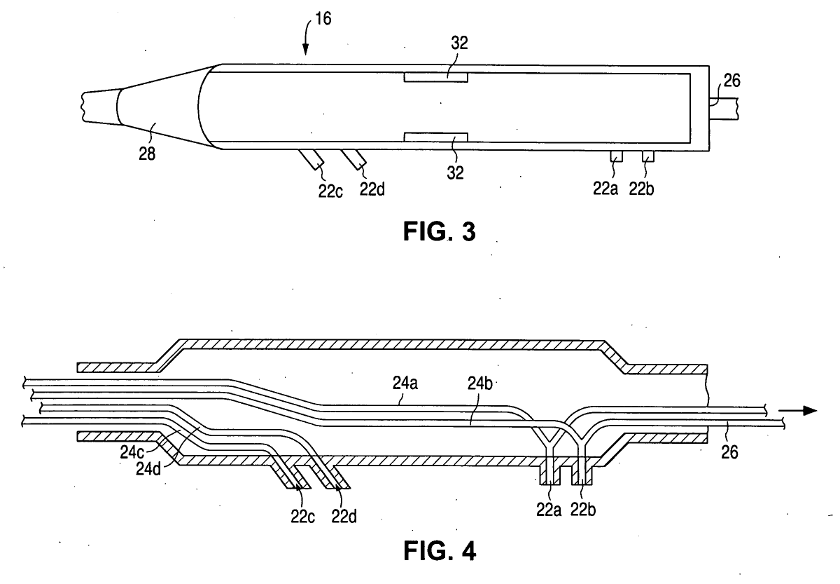

As best illustrated by FIGS. 1, 2 and 3, the stylet control handle receiving body 16 is generally tubular shape with a cutout on one side thereof. The cut out is large enough so as to allow, on one hand, a tight fit between the control handle 18 and the receiving body 16 and on the other hand allow for easy disengagement of the control handle 18 from the receiving body 16. The control handle receiving body 16 should be sized so that the device can be easily used by the physician. The receiving body 16 includes multiple ports and valves 22a-22d which can be in fluid communication with catheter lumens 24a-24d (see FIG. 4). These lumens 24a-24d can extend through or within the receiving body 16 and into the catheter tube 14 so as to prevent exposure of the control handle 18 and stylet member 20 to anything that is passed into and out of the lumens 24a-24d, including bodily fluids, air, water, and therapeutic or diagnostic tools. In some embodiments, one of the lumens can be used for a wash channel in that pressurized water can be injected through a nozzle at a distal end of the catheter tube 14 onto a lens covering for cleaning. Another channel can be used to apply a gas, such as air or CO₂ into the organ. Lumens can also be used to extract fluids or inject fluids, such as water or drugs in a liquid carrier, into the body. Various biopsy, drug delivery, balloon catheters and other devices which can be diagnostic and/or therapeutic in nature can also be inserted via the lumens to perform specific functions. In some embodiments, various tools include a retractable needle for drug injection, hydraulically actuated scissors, clamps, grasping tools, electrocoagulation systems, ultrasound transducers, electrical sensors, heating elements, other ablation means, etc. In some embodiments, at least two channels are provided to accommodate a diagnostic and/or therapeutic tool simultaneously. In one example, the physician has the capability of applying a drug to a lesion immediately after or contemporaneously with tissue extraction. Examples of other applicable devices include optical and image capturing devices, lasers, Doppler flow-meters, illuminating devices, etc.

Although four ports and valves 22 are illustrated, any number can be used. The only compromise with the use of a multitude of ports and valves 22 is that each can correspond with a lumen that extends through the catheter tube 14. As a result, size considerations must be taken into account as additional catheter lumens may compromise the functionality of the device. “Y” junctions can be used to designate two ports to a single lumen or one port to two lumens. In one embodiment, ports and valves 22a-22d correspond to air/water valve, suction valve, instrument channel, and suction/instrument channel, respectively. As is known to one of ordinary skill in the art, button/valve system can also be used such that depression of a top and a lower button (not illustrated) can provide for multiple functions through the same lumen or exchange of functions. An accessory outlet 26 allows for air/water 22a and suction valves 22b to be in fluid communication with pumps and related accessories. The control handle 18 also includes an accessory outlet (not shown) for a light source to apply light via the control handle 18 to distal end of the stylet member 20 and allows for optical or visual devices (e.g., lens and single chip sensor/multiple chip sensor), at the distal end of the stylet member 20, to be in communication with a processor.

The control handle receiving body 16 has a tubular body that extends into a generally conical distal end tip 28 that allows for insertion and engagement of the control handle 18 within the receiving body 16. The cutout portion of the receiving body 16 does not extend completely into this conically distal tip 28 so as to provide an overhang in which the control handle 18 can be inserted. The steerable stylet member 20 is first inserted into the catheter body 14 and snaked down into the catheter body 14 until a substantial length, preferably all, of the stylet member 20 is disposed in the catheter body 14. The control handle 18, having its own respective generally conical shaped distal tip 30 is slanted such that distal tip 30 is slid into the overhang of the distal tip 28 of the receiving body 16. The body of the control handle 18 is then inserted into the slot of the receiving body 16 in a friction fit type manner. The receiving body 16 can include opposing tabs 32 extending out from the ledges of the body 16 to allow for a “snap” action of the control handle 18 into the receiving body 16. In one embodiment, the tabs 32 include curved inner surfaces such that the surface of the tabs 32 can be in flush contact with and can compress against the control handle 18 when the control handle 18 is disposed in the receiving body 16. In some embodiments, the tabs 32 need to be tapered inwardly at a sufficient angle and spaced from one another to a degree such that the force of the control handle 18 caused the tab 32 to push outwardly and away from each other. The tabs 32 have to be flexible enough to rebound back into position for locking the control handle 18 into the receiving body 16. If the angle is to too pitched towards each other, insertion of the control handle 18 into the receiving body 16 may be difficult or the tabs 32 might break off during application. If the angle of the tabs 32 is, conversely, too great, it may not properly function to lock the control handle 18 into the receiving body 16.

The control handle 18 can be securely mated with or locked within the receiving body 16 and can be removed by pulling first on the proximal end of the control handle 18. Once the proximal end of the control handle 18 is disengaged, the distal tip 30 of the control handle 18 can be removed from the distal tip 28 of the receiving body 16 and the stylet member 20 extracted out from the lumen of the catheter tube 14. Care should be taken to ensure that the stylet 12 is not contaminated during the removal process.

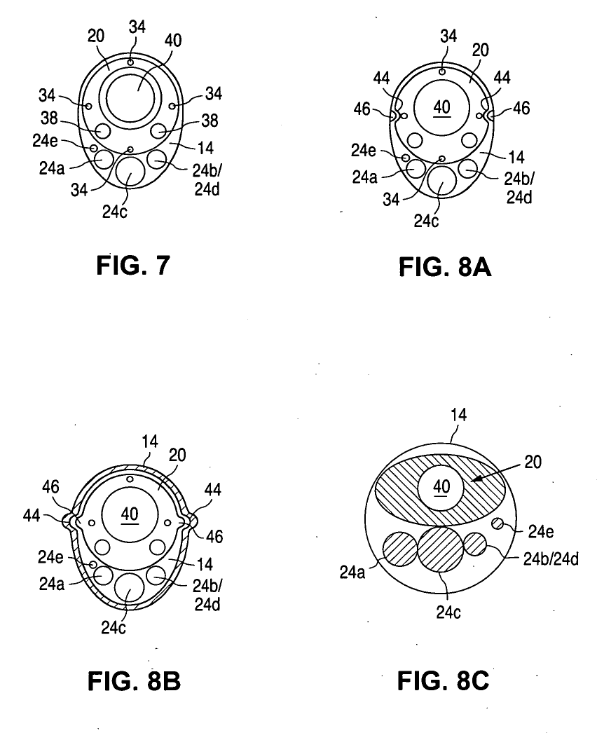

Manipulation of the stylet member 20 can be accomplished by retraction and extension of control cables or steering wires 34 (FIG. 7) symmetrically positioned within a core of the stylet member 20. The control cables 34 are anchored at or near the distal end of the core and extend along the length of the stylet member 20 to control knobs 36 that are rotated to move the distal end of the stylet member 20 in opposing directions. Four wires 34 and two knobs 36 account for up and down as well as side to side movement of the stylet member 20. The operation of a combination of two adjacent wires can provide for angular movement of the stylet member 20. In some embodiments, a clutch or breaking component could be included with the control knobs 36 as to preventing the knobs 36 from inadvertently rotating such that rotation can only be caused by application of a certain degree of torque to the knobs 36. Control knobs and clutches are well known by one having ordinary skill in the art.

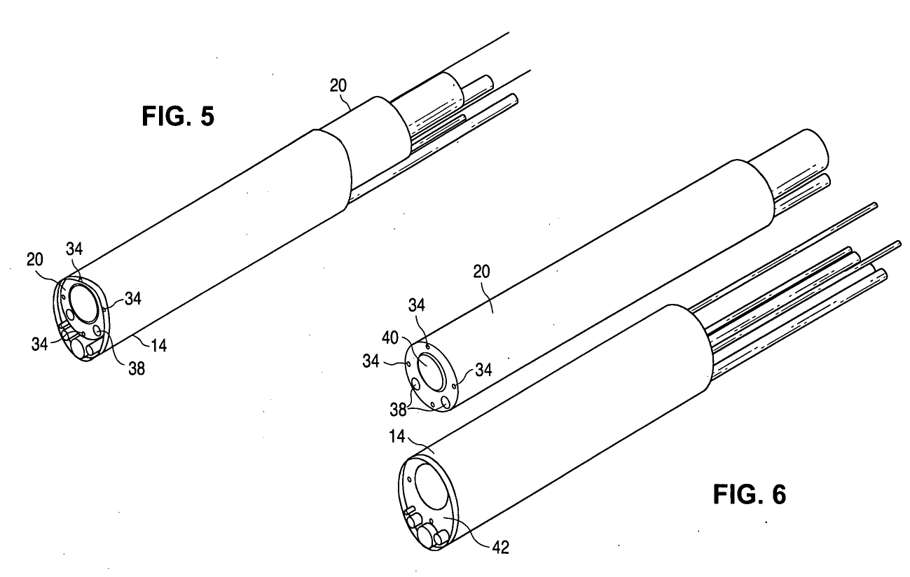

Referring now to FIGS. 5, 6, and 7 there is illustrated a distal portion of the steerable stylet member 20 disposed within the catheter tube 14, a disassembled stylet member 20 next to the catheter tube 14, and a cross section of the assembly. The stylet member 20 includes the control cables 34 extending and coupled therein as well as light emitting diodes (LEDs) 38 and a imaging or visual device 40. The visual or imaging device 40 can include a lens and single chip sensor or multiple chip sensor, for example. The visual or imaging device 40, in electrical communication with a processor, can be for taking single images or recorded or live video images. Control knobs 41 (FIG. 1) at proximal end of control handle 18 are for controlling image functions, such as focus, brightness, sharpness, etc. Control knobs 41 can also be used for turning the LEDs 38 on and off and controlling the intensity of the LEDs 38. The intensity of the LEDs 38 can be controlled individually so as to provide for optimum lighting. The LEDs 38 are strategically positioned between the outlet of lumens 24 for the instrumentation(s) and the lens 40 so as to provide for adequate lighting. As is best illustrated by FIG. 7, in some embodiments, lumens 24b and 24d can share the same exit nozzle in that the lumens include a “Y” connector. An added exit nozzle, 24e, is also illustrated in this figure. The nozzle 24e can be a water jet channel.

In some embodiment the stylet member 20 can be made from a material so as to allow a user to be able to easily thread the stylet member 20 within the lumen of the catheter tube 14. Examples of suitable material include pTFE, PVC, polyurethane and various known elastomers. In some embodiments, a certain degree of friction is required between the outer surface of the stylet member 20 and the inner surface of the catheter tube 14 so as to prevent the stylet member 20 from getting inadvertently rotated within the catheter body 14 during a procedure. In some embodiments a groove—railing combination can be provided so as to prevent the stylet member 20 from rotating within the catheter tube 14. This combination is illustrated by FIGS. 8A and 8B. FIG. 8A illustrates a cross section of the stylet member 20 inserted within the catheter tube 14. The stylet member 20 includes an opposing pair of longitudinally extending channels 44 disposed along the length of the outer surface of the stylet member 20. The channels 44 can start from the distal tip of the stylet member 20 and extend at least to the proximal end of the stylet member 20. The catheter tube 14 includes an opposing pair of protruding rails 46 that extend longitudinally along an inner surface of the catheter tube 14 so as to mate with their respective channels 44. The stylet member 20 is inserted into the catheter tube 14 such that the grooves 44 are directed over the rails 46. The stylet member 20 is then threaded down into the catheter tube 14. The groove-rail assembly prevents rotation of the stylet member 20 in the catheter tube 14. FIG. 8B illustrates a configuration in which the outer surface of the stylet member 20 includes the rail 46 and the inner surface of the catheter tube includes the channel 44. Other means of preventing rotation of one member with respect to the other or for transmission of torque are equally applicable as well, such as non-circular stylet member 20 disposed in a non-circular lumen, having the same or different cross sectional shape as the stylet member 20. In some embodiments, as illustrated by FIG. 8C, the stylet member 20 is oval shaped and received in an oval shaped lumen so as to prevent the rotation of the stylet member with respect to the catheter tube 14.

Preferred dimensions for the stylet include 6 to 9 mm in diameter and 1 to 2 meters in length (depending on its use, e.g., upper or lower GI).

The catheter tube 14 includes, as discussed above, multiple lumens 24a-24d for performing various functions. The functions include ablation, drug delivery, biopsy, infusion of water or a gas, extraction of fluids, application of fluids, imaging, etc. The catheter tube 14 also includes a lumen for receiving the stylet member 20. The end of the catheter tube 14 as best illustrated by FIG. 6, is enclosed such that only the lumens 24a-d are exposed to tissues, organs and bodily fluids. To allow the camera 40 to capture images, the catheter tube 14 is enclosed with a transparent plate or window 42. In essence, the transparent window 42 caps the lumen in which the stylet member 20 is disposed. The catheter tube 14 can be made from conventional catheter material known to one having ordinary skill in the art. The dimensions of the catheter tube 14 can be 6 to 13 mm, more narrowly 9 to 12 mm in diameter and 1 to 2 meters long.

The multi-lumen disposable catheter with a reusable stylet is preferably intended for the following procedures: anoscopy, arthroscopy, bronchoscopy, colonoscopy, cytoscopy, esophagogastroduodenoscopy (EGD), ERCP, laparoscopy, sigmoidoscopy, and examination of the small intestine. However, its use is not intended to be limited to these applications as it can be used in any medical application in which a scope is needed.

In some embodiments, the same design concept can be used but for the fact that both the catheter assembly 10 and the stylet 12 are fully disposable. The control handle 18 can be of one part with the receiving body 16. Accordingly, the assembly is made from a single component of catheter-stylet. Alternatively, the control handle 18 of the stylet 12 can be separable from the catheter assembly 10, but still fully disposable in kind.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A disposable catheter and reusable stylet assembly, comprising:
(a) a catheter assembly comprising
(i) a catheter tube having a first lumen and a second lumen extending at least partially through the catheter tube; and
(ii) a stylet receiving body connected to the catheter tube, the receiving body including an opening along a longitudinal side of the body and a port disposed within the receiving body and in fluid communication with the first lumen; and
(b) a stylet comprising
(i) a control handle capable of being securely inserted in the longitudinal side opening of the receiving body;
(ii) a stylet member for being extended through the second lumen of the catheter assembly; and
(iii) a visual or image device including components positioned at a distal end of the stylet member.

2. The disposable catheter and reusable stylet assembly of claim 1, wherein the catheter assembly prevents the stylet member from making contact with bodily fluids or tissues.

3. The disposable catheter and reusable stylet assembly of claim 1, wherein the control handle disengagably locks within the receiving body.

4. The disposable catheter and reusable stylet assembly of claim 1, additionally comprising a pair of opposing tabs extending from the receiving body to securely lock the control handle within the receiving body.

5. The disposable catheter and reusable stylet assembly of claim 1, wherein the receiving body includes an overhang, at an end thereof, in which a distal end of the control handle is inserted for engagement of the control handle to the receiving body.

6. The disposable catheter and reusable stylet assembly of claim 5, wherein the overhang prevents the control handle from being coupled to the receiving body unless a distal end of the control handle is first inserted into the overhang.

7. The disposable catheter and reusable stylet assembly of claim 1, wherein at a distal end of the catheter tube, the second lumen is covered by a transparent plate so as to allow the visual or image device to capture images and further to prevent the stylet member from making contact with bodily fluids or tissues.

8. The disposable catheter and reusable stylet assembly of claim 7, wherein the first lumen is exposed to bodily fluids or tissues at a distal end of the catheter tube.

9. The disposable catheter and reusable stylet assembly of claim 1, additionally comprising one or more functional lumens disposed in the catheter tube and in communication with respect port(s) of the stylet receiving body for receiving diagnostic and/or therapeutic tool(s).

10. The disposable catheter and reusable stylet assembly of claim 1, wherein the receiving body includes a generally tubular shape having the opening on one side of the body and a coning end portion extending from an end of the receiving body and wherein the control handle includes a generally coning end portion configured to be fittingly mated within the end of the receiving body.

11. The disposable catheter and reusable stylet assembly of claim 1, wherein the control handle and the receiving body are securely engagable via a friction fit.

12. The disposable catheter and reusable stylet assembly of claim 1, wherein the visual or image device includes a lens and a single chip sensor or multiple chip sensors.

13. The disposable catheter and reusable stylet assembly of claim 1, wherein the visual or image device can capture single images or recorded or live video.

14. The disposable catheter and reusable stylet assembly of claim 1, wherein the stylet additionally comprises means for controlling or causing the movement of the stylet member.

15. The disposable catheter and reusable stylet assembly of claim 1, wherein the stylet member has a non-circular cross section.

16. The disposable catheter and reusable stylet assembly of claim 1, wherein the stylet member and/or the second lumen have non-circular cross sectional shapes.

17. The disposable catheter and reusable stylet assembly of claim 1, wherein the stylet member has an oval cross sectional shape.

18. The disposable catheter and reusable stylet assembly of claim 1, wherein the stylet additionally comprises rotatable control knobs on the control handle and cables, connected to the control knobs and extending along the stylet member for directing a movement of the stylet member.

19. A method of performing an ablation, biopsy and/or drug delivery procedure, comprising inserting the device of claim 1 into a bodily lumen or cavity of a patient followed by performing an ablation, biopsy and/or drug delivery procedure.

20. The method of claim 19, wherein the bodily lumen or cavity is esophagus, stomach, duodenum, colon, bronchi, peritoneal cavity, rectum, sigmoid colon or a blood vessel.
